(19) United States
(12) Patent Application Publication
(10) Pub. No.: US 2003/0008869 A1
(43) Pub. Date: Jan. 9, 2003

(54) Combination of a PTPase inhibitor and a sulfonylurea agent

(75) Inventors: Gayle Penniman Orczyk, Berwyn, PA (US); John Thomas Gwynne, Doylestown, PA (US)

Correspondence Address: Arnold S. Milowsky, 5 Giralda Farms, Madison, NJ, 07940, US

(73) Assignee: Wyeth, Madison, NJ

(21) Appl. No.: 10/163,783

(22) Filed: Jun. 6, 2002

Related U.S. Application Data
(60) Provisional application No. 60/296,494, filed on Jun. 7, 2001.

(51) Int. Cl. A61K 31/5377; A61K 31/4439; A61K 31/405; A61K 31/381; A61K 31/343

(52) U.S. Cl. 514/232.8; 514/337; 514/339; 514/415; 514/443; 514/469

(57) ABSTRACT

This invention provides pharmaceutical compositions and methods of treatment and control of Syndrome X or type II diabetes in a mammal utilizing a combination of a sulfonylurea agent and a protein-tyrosine phosphatase (PTPase) inhibitors.

This application claims priority from copending provisional application Serial No. 60/296,494, filed Jun. 7, 2001, the entire disclosure of which is hereby incorporated by reference.

This invention provides pharmaceutical compositions and methods of treatment utilizing a combination of a protein-tyrosine phosphatase (PTPase) inhibitors and a sulfonylurea agent of use in treatment, inhibition control or maintenance of Syndrome X or type II diabetes in a mammal.

### BACKGROUND OF THE INVENTION

The prevalence of insulin resistance in glucose intolerant subjects has long been recognized. Reaven et al (American Journal of Medicine 1976, 60, 80) used a continuous infusion of glucose and insulin (insulin/glucose clamp technique) and oral glucose tolerance tests to demonstrate that insulin resistance existed in a diverse group of nonobese, nonketotic subjects. These subjects ranged from borderline glucose tolerant to overt, fasting hyperglycemia. The diabetic groups in these studies included both insulin dependent (IDDM) and noninsulin dependent (NIDDM) subjects.

Coincident with sustained insulin resistance is the more easily determined hyperinsulinemia, which can be measured by accurate determination of circulating plasma insulin concentration in the plasma of subjects. Hyperinsulinemia can be present as a result of insulin resistance, such as is in obese and/or diabetic (NIDDM) subjects and/or glucose intolerant subjects, or in IDDM subjects, as a consequence of over injection of insulin compared with normal physiological release of the hormone by the endocrine pancreas.

The association of hyperinsulinemia with obesity and with ischemic diseases of the large blood vessels (e.g. atherosclerosis) has been well established by numerous experimental, clinical and epidemiological studies (summarized by Stout, Metabolism 1985, 34, 7, and in more detail by Pyorala et al, Diabetes/Metabolism Reviews 1987, 3, 463). Statistically significant plasma insulin elevations at 1 and 2 hours after oral glucose load correlates with an increased risk of coronary heart disease.

Since most of these studies actually excluded diabetic subjects, data relating the risk of atherosclerotic diseases to the diabetic condition are not as numerous, but point in the same direction as for nondiabetic subjects (Pyorala et al). However, the incidence of atherosclerotic diseases in morbidity and mortality statistics in the diabetic population exceeds that of the nondiabetic population (Pyorala et al; Jarrett Diabetes/Metabolism Reviews 1989,5, 547; Harris et al, Mortality from diabetes, in Diabetes in America 1985).

The independent risk factors obesity and hypertension for atherosclerotic diseases are also associated with insulin resistance. Using a combination of insulin/glucose clamps, tracer glucose infusion and indirect calorimetry, it has been demonstrated that the insulin resistance of essential hypertension is located in peripheral tissues (principally muscle) and correlates directly with the severity of hypertension (DeFronzo and Ferrannini, Diabetes Care 1991, 14, 173). In hypertension of the obese, insulin resistance generates hyperinsulinemia, which is recruited as a mechanism to limit further weight gain via thermogenesis, but insulin also increases renal sodium reabsorption and stimulates the sympathetic nervous system in kidneys, heart, and vasculature, creating hypertension.

It is now appreciated that insulin resistance is usually the result of a defect in the insulin receptor signaling system, at a site post binding of insulin to the receptor. Accumulated scientific evidence demonstrating insulin resistance in the major tissues which respond to insulin (muscle, liver, adipose), strongly suggests that a defect in insulin signal transduction resides at an early step in this cascade, specifically at the insulin receptor kinase activity, which appears to be diminished (reviewed by Haring, Diabetalogia 1991, 34, 848).

Protein-tyrosine phosphatases (PTPases) play an important role in the regulation of phosphorylation of proteins. The interaction of insulin with its receptor leads to phosphorylation of certain tyrosine molecules within the receptor protein, thus activating the receptor kinase. PTPases dephosphorylate the activated insulin receptor, attenuating the tyrosine kinase activity. PTPases can also modulate post-receptor signaling by catalyzing the dephosphorylation of cellular substrates of the insulin receptor kinase. The enzymes that appear most likely to closely associate with the insulin receptor and therefore, most likely to regulate the insulin receptor kinase activity, include PTP1 B, LAR, PTPα and SH-PTP2 (B. J. Goldstein, J. Cellular Biochemistry 1992, 48, 33; B. J. Goldstein, Receptor 1993, 3, 1-15,; F. Ahmad and B. J. Goldstein Biochim. Biophys Acta 1995, 1248, 57-69).

McGuire et al. (Diabetes 1991, 40, 939), demonstrated that nondiabetic glucose intolerant subjects possessed significantly elevated levels of PTPase activity in muscle tissue vs. normal subjects, and that insulin infusion failed to suppress PTPase activity as it did in insulin sensitive subjects.

Meyerovitch et al (J. Clinical Invest. 1989, 84, 976) observed significantly increased PTPase activity in the livers of two rodent models of IDDM, the genetically diabetic BB rat, and the STZ-induced diabetic rat. Sredy et al (Metabolism, 44, 1074, 1995) observed similar increased PTPase activity in the livers of obese, diabetic ob/ob mice, a genetic rodent model of NIDDM.

The compounds of us in the methods of this invention have been shown to inhibit PTPases derived from rat liver microsomes and human-derived recombinant PTPase-1B (hPTP-1B) in vitro. Their synthesis and use in treatments of insulin resistance associated with obesity, glucose intolerance, diabetes mellitus, hypertension and ischemic diseases of the large and small blood vessels is taught in published PCT Application WO 99/61435 (Wrobel et al.).

### DESCRIPTION OF THE INVENTION

This invention provides methods of using PTPase inhibitors and a sulfonylurea agent for the management of Syndrome X or type 2 diabetes and for improving the cardiovascular risk profile in mammals experiencing or subject to those maladies. These methods may also be characterized as the reduction of risk factors in such mammals for heart disease, stroke or heart attack in a type II diabetic.

These methods include the reduction of hyperlipidemia in a mammal experiencing or subject to Syndrome X or type II diabetes, including methods for lowering low density lipoprotein (LDL) blood levels, high density lipoprotein (HDL) blood levels, and overall blood lipoprotein levels in such mammals. The methods herein may further be characterized as inhibiting, preventing or reducing atherosclerosis in a mammal subject to or experiencing Syndrome X or type II diabetes, or the risk factors thereof.

These methods also include the lowering free fatty acid blood levels and triglyceride levels in such mammals.

The methods of this invention comprise administering to a mammal in need thereof a pharmaceutically effective amount of a PTPase inhibitor and a pharmaceutically effective amount of a sulfonylurea agent.

Sulfonylurea agents useful with the methods and compositions of this invention include glipizide, glyburide (glibenclamide), chlorpropamide, tolbutamide, tolazamide and glimepriride, or the pharmaceutically acceptable salt forms thereof.

Each of these methods and compositions may utilize a pharmaceutically effective amount of a PTPase inhibiting compound of formula I:

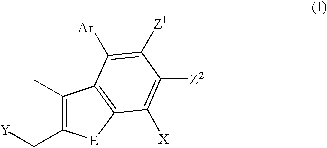

wherein:

Ar is

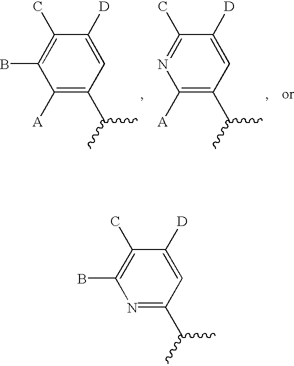

A is hydrogen, halogen, or OH;

B and D are each, independently, hydrogen, halogen, CN, alkyl of 1-6 carbon atoms, aryl, aralkyl of 6-12 carbon atoms, hydroxyalkyl of 1-6 carbon atoms, hydroxyaralkyl of 6-12 carbon atoms, cycloalkyl of 3-8 carbon atoms, nitro, amino, —NR¹R^{1a}, —NR¹COR^{1a}, —NR¹CO₂R^{1a}, cycloalkylamino of 3-8 carbon atoms, morpholino, furan-2-yl, furan-3-yl, thiophen-2-yl, thiophen-3-yl, —COR^{1b} or OR;

R is hydrogen, alkyl of 1-6 carbon atoms, —COR¹, —(CH₂)ₙCO₂R¹, —CH(R^{1a}) CO₂R¹, —SO₂R¹, —(CH₂)ₘCH(OH)CO₂R¹, —(CH₂)ₘCOCO₂R¹, —(CH₂) ₘCH═CHCO₂R¹, or —(CH₂)ₘO(CH₂)₀CO₂R¹;

R¹ is hydrogen, alkyl of 1-6 carbon atoms, aralkyl of 6-12 carbon atoms, aryl, or CH₂CO₂R^{1′};

R^{1′}is hydrogen or alkyl of 1-6 carbon atoms E is S, SO, SO₂, O, or NR^{1c};

X is hydrogen, halogen, alkyl of 1-6 carbon atoms, alkenyl of 2-7 carbon atoms, CN, aryl, aralkyl of 6-12 carbon atoms, hydroxyalkyl of 1-6 carbon atoms, hydroxyaralkyl of 6-12 carbon atoms, perfluoroalkyl of 1-6 carbon atoms, alkoxy of 1-6 carbon atoms, aryloxy; arylalkoxy, nitro, amino, NR²R²a, NR²COR^{2a}, cycloalkylamino of 3-8 carbon atoms, morpholino, alkylsulfanyl of 1-6 carbon atoms, arylsulfanyl, pyridylsulfanyl, 2-N,N-dimethylaminoethylsulfanyl, —OCH₂CO₂R^{2b} or —COR^{2c};

Y is hydrogen, halogen, alkyl of 1-6 carbon atoms, aryl, aralkyl of 6-12 carbon atoms, hydroxyalkyl of 1-6 carbon atoms, hydroxyaralkyl of 6-12 carbon atoms, —OR³, SR³, NR³R^{3a}, —COR^{3b}, morpholine or piperidine;

R^{1a}, R^{1c}, R², R^{2a} R³, R^{3a} are each, independently, hydrogen, alkyl of 1-6 carbon atoms, aralkyl of 6-12 carbon atoms, or aryl;

R^{1b} is alkyl of 1-6 carbon atoms or aryl;

R^{2b} is hydrogen, alkyl of 1-6 carbon atoms;

R^{2c} and R^{3b} are each, independently, alkyl of 1-6 carbon atoms, aryl, or aralkyl of 6-12 carbon atoms;

C is hydrogen, halogen or OR⁴;

R⁴ is hydrogen, alkyl of 1-6 carbon atoms, —CH(R₅)W, —C(CH₃)₂CO₂R⁶, 5-thiazolidine-2,4-dione, —CH(R⁷)(CH₂)ₘCO₂R⁶, —COR⁶, —PO₃(R⁶)₂, —SO₂R⁶, —(CH₂)ₚCH(OH)CO₂R⁶, —(CH₂)ₚCOCO₂R⁶, —(CH₂)ₚCH═CHCO₂R⁶, or —(CH2)ₚO(CH₂)_{q}CO₂R⁶;

R⁵ is hydrogen, alkyl of 1-6 carbon atoms, aralkyl of 6-12 carbon atoms, aryl, —CH₂(1H-imidazol-4-yl), —CH₂(3-1H-indolyl), —CH₂CH₂(1,3-dioxo-1,3-dihydro-isoindol-2-yl), —CH₂CH₂(1-oxo-1,3-dihydro-isoindol-2-yl), —CH₂(3-pyridyl), —CH₂CO₂H, or —(CH₂)ₙG;

G is NR^{6a}R^{7a}, NR^{6a}COR^{7a}

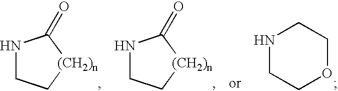

W is CO₂R⁶, CONH₂, CONHOH, CN, CONH(CH₂)₂CN, 5-tetrazole, —PO₃(R⁶)₂, —CH₂OH, —CONR^{6b}CHR^{7b}, —CH₂NR^{6b}CHR^{7b}CO₂R⁶, —CH₂OCHR^{7b}CO₂R⁶ —CH₂Br, or —CONR^{6b}CHR^{7b}CO₂R⁶;

R⁶, R^{6a}, R⁷, R^{7a} are each, independently, is hydrogen, alkyl of 1-6 carbon atoms, or aryl;

R^{6b} is hydrogen or —COR^{6c};

R^{6c} is alkyl of 1-6 carbon atoms or aryl;

R^{7b} is hydrogen, alkyl of 1-6 carbon atoms, or hydroxyalkyl of 1-6 carbon atoms;

Z¹ and Z² are each, independently, hydrogen, halogen, CN, alkyl of 1-6 carbon atoms, aryl, aralkyl of 6-12 carbon atoms, cycloalkyl of 3-8 carbon atoms, nitro, amino, —NR¹R^{1a}, —-NR¹COR^{1a}, cycloalkylamino of 3-8 carbon atoms, morpholino, or OR⁸, or Z¹ and Z² may be taken together as a diene unit having the formula —CH═CR⁹—CR¹⁰═CR¹¹—;

R⁸ is hydrogen, alkyl of 1-6 carbon atoms, or aryl;

R⁹, R¹⁰, and R¹¹ are each, independently, hydrogen, alkyl of 1-6 carbon atoms, aryl, halogen, hydroxy, or alkoxy of 1-6 carbon atoms

m is 1 to 4

n is 1 or 2;

p is 1 to 4;

q is 1 to 4;

or a pharmaceutically acceptable salt or ester form thereof.

The synthesis and PTPase inhibiting and anti-diabetic activities of the compounds described herein are demonstrated in published PCT Application WO 99/61435 (Wrobel et al.), published Dec. 2, 1999, the contents of which are incorporated herein by reference.

Pharmaceutically acceptable salts of these compounds can be formed from organic and inorganic acids, for example, acetic, propionic, lactic, citric, tartaric, succinic, fumaric, maleic, malonic, mandelic, malic, phthalic, hydrochloric, hydrobromic, phosphoric, nitric, sulfuric, methanesulfonic, napthalenesulfonic, benzenesulfonic, toluenesulfonic, camphorsulfonic, and similarly known acceptable acids when a compound of this invention contains a basic moiety, such as when R⁵ is CH₂(3-pyridyl), or Y is morpholine or contains similar basic moieties. Salts may also be formed from organic and inorganic bases, preferably alkali metal salts, for example, sodium, lithium, or potassium, when a compound of this invention contains a carboxylate or phenolic moiety.

Alkyl includes both straight chain as well as branched moieties. Halogen means bromine, chlorine, fluorine, and iodine. It is preferred that the aryl portion of the aryl or aralkyl substituent is a phenyl or naphthyl; with phenyl being most preferred. The aryl moiety may be optionally mono-, di-, or tri- substituted with a substituent selected from the group consisting of alkyl of 1-6 carbon atoms, alkoxy of 1-6 carbon atoms, trifluoromethyl, halogen, alkoxycarbonyl of 2-7 carbon atoms, alkylamino of 1-6 carbon atoms, and dialkylamino in which each of the alkyl groups is of 1-6 carbon atoms, nitro, cyano, —CO₂H, alkylcarbonyloxy of 2-7 carbon atoms, and alkylcarbonyl of 2-7 carbon atoms.

The PTPase inhibiting compounds used in the methods of this invention may contain an asymmetric carbon atom and some of the compounds of this invention may contain one or more asymmetric centers and may thus give rise to optical isomers and diastereomers. While shown without respect to stereochemistry in Formula I, the present invention includes such optical isomers and diastereomers; as well as the racemic and resolved, enantiomerically pure R and S stereoisomers; as well as other mixtures of the R and S stereoisomers and pharmaceutically acceptable salts thereof.

The compounds of this invention may be atropisomers by virtue of possible restricted or slow rotation about the aryl-tricyclic or aryl-bicyle single bond. This restricted rotation creates additional chirality and leads to enantiomeric forms. If there is an additional chiral center in the molecule, diasteriomers exist and can be seen in the NMR and via other analytical techniques. While shown without respect to atropisomer stereochemistry in Formula I, the present invention includes such atoropisomers (enantiomers and diastereomers; as well as the racemic, resolved, pure diastereomers and mixtures of diasteomers) and pharmaceutically acceptable salts thereof.

Preferred PTPase compounds of use in this invention include those having the structure:

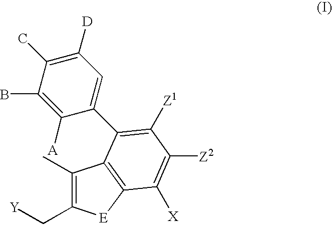

wherein:

A is hydrogen or halogen;

B and D are each, independently, hydrogen, halogen, CN, alkyl of 1-6 carbon atoms, aryl, aralkyl of 6-12 carbon atoms, branched alkyl, cycloalkyl of 3-8 carbon atoms, nitro or OR;

R is hydrogen or alkyl of 1-6 carbon atoms;

E is S, or O;

X is hydrogen, halogen, alkyl of 1-6 carbon atoms, CN, perfluoroalkyl of 1-6 carbon atoms, alkoxy of 1-6 carbon atoms, aryloxy; arylalkoxy, nitro, amino, NR²R^{2a}, NR²COR^{2a}, cycloalkylamino, morpholino, alkylsulfanyl of 1-6 carbon atoms, arylsulfanyl, pyridylsulfanyl, 2-N,N-dimethylaminoethylsulfanyl;

R¹, R^{1a}, R², R^{2a}, R³, and R^{3a} are each, independently, hydrogen, alkyl of 1-6 carbon atoms, aralkyl of 6-12 carbon atoms, or aryl;

Y is hydrogen, halogen, OR³, SR³, NR³R^{3a} or morpholine;

C is hydrogen, halogen, or OR⁴;

R⁴ is hydrogen, alkyl of 1-6 carbon atoms, —CH(R⁵)W, —C(CH₃)₂CO₂R⁶, 5-thiazolidine-2,4-dione, —CH(R⁷)(CH₂)ₘCO₂R⁶, —COR⁶, —PO₃(R⁶) ₂, —SO₂R⁶, —(CH₂)ₚCH(OH)CO₂R⁶, —(CH₂)ₚCOCO₂R⁶, —(CH₂)ₚCH═CHCO₂R⁶, or —(CH₂)ₚO(CH₂)_{q}CO₂R⁶;

R⁵ is hydrogen, alkyl of 1-6 carbon atoms, aralkyl of 6-12 carbon atoms, aryl, —CH₂(1H-imidazol-4-yl), —CH₂(3-1H-indolyl), —CH₂CH₂(1,3-dioxo-1,3-dihydro-isoindol-2-yl), —CH₂CH₂(1-oxo-1,3-dihydro-isoindol-2-yl), or —CH₂(3-pyridyl);

W is CO₂R⁶, —CONH₂, —CONHOH, or 5-tetrazole, or —CONR^{6b}CHR^{7b}CO₂R⁶;

R⁶, R^{6a}, R^{6b},R⁷, R^{7a}, and R^{7b} are each, independently, hydrogen, alkyl of 1-6 carbon atoms, or aryl;

Z¹ and Z² are each, independently, hydrogen, halogen, CN, alkyl of 1-6 carbon atoms, aryl, aralkyl of 6-12 carbon atoms, cycloalkyl of 3-8 carbon atoms, nitro, amino, —NR¹R^{1a}, —NR¹COR^{1a}, cycloalkylamino of 3-8 carbon atoms, morpholino, or OR⁸, or Z¹ and Z² may be taken together as a diene unit having the formula —CH═CR⁹—CR¹⁰═CH—;

R⁹ and R¹⁰ are independently, hydrogen, or alkyl of 1-6 carbon atoms;

p is 1 to 4;

q is 1 to 4;

or a pharmaceutically acceptable salt or ester form thereof.

More preferred PTPase inhibiting compounds for use with this invention include those of the structure:

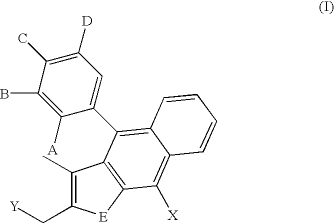

wherein:

A is hydrogen;

B and D are each, independently, halogen, alkyl of 1-6 carbon atoms, aryl, aralkyl of 6-12 carbon atoms, or cycloalkyl of 3-8 carbon atoms;

E is S or O;

X is hydrogen, halogen, alkyl of 1-6 carbon atoms, perfluoroalkyl of 1-6 carbon atoms, CN, alkoxy of 1-6 carbon atoms, aryloxy, arylalkoxy of 6-12 carbon atoms, arylsulfanyl;

Y is hydrogen or —NR¹R², or morpholine;

R¹ and R² are each, independently, hydrogen or alkyl of 1-6 carbon atoms, aralkyl of 6-12 carbon atoms, or aryl;

C is OR⁴;

R⁴ is hydrogen, alkyl of 1-6 carbon atoms, —CH(R⁵)W, or 5-thiazolidine-2,4-dione;

R⁵ is hydrogen, alkyl of 1-6 carbon atoms, aralkyl of 6-12 carbon atoms, aryl, —CH₂(3-1H-indolyl), —CH₂CH₂(1,3-dioxo-1,3-dihydro-isoindol-2-yl), or —CH₂CH₂(1-oxo-1,3-dihydro-isoindol-2-yl);

W is —CO₂R⁶, —CONH₂, —CONHOH, 5-tetrazole, —PO₃(R⁶)₂, or —CONR⁶CHR⁶CO₂R⁶

R⁶ is hydrogen or alkyl of 1-6 carbon atoms;

Z¹ and Z² are taken together as a diene unit having the formula —CH═CH—H═CH—;

or a pharmaceutically acceptable salt thereof.

Even more preferred PTPase inhibitor compounds of this invention include:

(R)-2-[2,6-dibromo-4(9-bromo-2,3-dimethyl-naphtho[2,3-b]thiophen-4-yl)-phenoxy]-3-phenyl-propionic acid;

(R)-2-[2-bromo-4—(9-bromo-2, 3-dimethyl-naphtho[2,3-b]thiophen-4-yl)-6-ethyl-phenoxy]-3-phenyl-propionic acid;

(R)-2-[4-(9-bromo-2, 3-dimethyl-naphtho[2,3-b]thiophen-4-yl)-2, 6-dimethyl-phenoxy]-3-phenyl-propionic acid;

(R)-2-[4(9-bromo-2, 3-dimethyl-naphtho[2,3-b]thiophen-4-yl)-2-fluoro-phenoxy]-3-phenyl-propionic acid;

[4-(9-bromo-2, 3-dimethyl-naphtho[2,3-b]thiophen-4-yl)-2, 6-diisopropyl-phenoxy]-acetic acid;

(R)-2-[2-bromo-4-(9-bromo-2, 3-dimethyl-naphtho[2,3-b]thiophen-4-yl)-6-sec-butyl-phenoxy]-3-phenyl-propionic acid;

(R)-2-[2-bromo-4-(9-bromo-2, 3-dimethyl-naphtho[2,3-b]thiophen-4-yl)-6-isopropyl-phenoxy]-3-phenyl-propionic acid;

(R)-2-[2-bromo-4-(9-bromo-2, 3-dimethyl-naphtho[2,3-b]thiophen-4-yl)-2-cyclopentyl-phenoxy]-3-phenyl-propionic acid

(R)-2-[4-(9-bromo-2, 3-dimethyl-naphtho[2,3-b]thiophen-4-yl)-6-isopropyl-phenoxy]-3-phenyl-propionic acid;

(R)-2-[4-(9-bromo-2, 3-dimethyl-naphtho[2,3-b]thiophen-4-yl)-2-cyclopentyl-phenoxy]-3-phenyl-propionic acid;

(R)-2-[2,6-dibromo-4-(2,3-dimethyl-9-phenylsulfanyl-naphtho[2,3-b]thiophen-4-yl)-phenoxy]-3-phenyl-propionic acid;

(R)-2-[2,6-dibromo-4-(9-bromo-2,3-dimethyl-naphtho[2,3-b]thiophen-4-yl)-phenoxy]-4-phenyl-butyric acid;

(S)-2-[2,6-dibromo-4-(9-bromo-2,3-dimethyl-naphtho[2,3-b]thiophen-4-yl)-phenoxy]-4-phenyl-butyric acid;

2-[2,6-dibromo-4-(9-bromo-3-methyl-2-morpholin-4-ylmethyl-naphtho[2,3-b]thiophen-4-yl)-phenoxy]-3-phenyl-propionic acid;

(R)-2-[2,6-dibromo-4-(2,3-dimethyl-9-phenylsulfanyl-naphtho[2,3-b]thiophen-4-yl)-phenoxy]-propionic acid;

[2-bromo-4-(9-bromo-2,3-dimethyl-naphtho[2,3-b]thiophen-4-yl)-2-nitro-phenoxy]-3-phenyl-propionic acid;

2, 6-dibromo-4-(9-bromo-2, 3-dimethyl-naphtho[2,3-b]thiophen-4-yl)-phenol;

2-bromo-4-(9-bromo-2, 3-dimethyl-naphtho[2,3-b]thiophen-4-yl)-2-nitro-phenol;

(R)-2-[2,6-dibromo-4-(9-bromo-2-diethylaminomethyl-3-methyl-naphtho[2,3-b]thiophen-4-yl)-phenoxy]-3-phenyl-propionic acid;

(R)-2-[2, 6-dibromo-4-(2, 3-dimethyl-naphtho[2,3-b]furan-4-yl)-phenoxy]-3-phenyl-propionic acid,

(2R)-2-[4-(9-bromo-2,3-dimethyl-naphtho[2,3-b]thiophen-4-yl)-2,6-diisopropyl-phenoxy]-3-phenyl-propionic acid,

(R)-2-[4-(9-bromo-2-,3-dimethyl-naphtho[2,3-b]thiophen-4-yl)-2,6-diethyl-phenoxy]-3-phenyl-propionic acid,

{(2R)-2-[4-(9-bromo-2,3-dimethyl-naphtho[2,3-b]thiophen-4-yl)-2,6-dimethyl-phenoxy]-3-phenyl-propionylamino}-acetic acid;

{(2R)-2-[4-(9-bromo-2,3-dimethyl-naphtho[2,3-b]thiophen-4-yl)-2,6-diethyl-phenoxy]-3-phenyl-propionylamino}-acetic acid

or pharmaceutically acceptable salts thereof.

Among the most preferred PTPase inhibiting compounds for use in the present inventions is (2R)-2-[4-(9-Bromo-2,3-dimethyl-naptho[2,3-b]thiophen-4-yl)-2,6-dimethyl-phenoxy]-3-phenyl-propionic acid, having the structure:

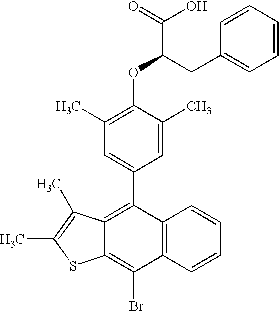

or its pharmaceutically acceptable salt or ester forms.

This invention provides methods for treating, preventing, inhibiting or ameliorating the basis or symptoms of various cardiovascular diseases in a mammal experiencing or subject to Syndrome X or type II diabetes, preferably in a human in need of such treatment. The methods each comprise administering to such a mammal in need thereof a pharmaceutically or therapeutically effective amount of a PTPase inhibitor of this invention and a sulfonylurea agent, as described herein. As used herein a pharmaceutically or therapeutically effective amount is understood to be at least a minimal amount which provides a medical improvement in the symptoms of the specific malady or disorder experienced by the mammal in question. Preferably, the recipient will experience a reduction, inhibition or removal of the biological basis for the malady in question.

A method of this invention comprises a reduction in the risk profile of cardiovascular diseases in such a mammal. This method may also be described as a method of inhibiting, preventing or reducing the physiological basis or causative elements of cardiovascular or cerebrovascular diseases in such mammals. These cardiovascular include atherosclerosis and coronary artery disease.

Another portion of this invention comprises a method of lowering blood cholesterol in a mammal experiencing or subject to Syndrome X or type II diabetes, the method particularly including reduction of lowering of low density lipoprotein (LDL) in such a mammal. Also provided is a method of lowering blood triglyceride levels in such a mammal. These actions may also be seen as a method for lowering the chances or risk of the mammal experiencing related cerebrovascular or cardiovascular disorders, including coronary artery disease (atherosclerosis), heart attack or stroke.

Effective administration of the PTPase inhibiting compounds herein may be given at a daily dosage of from about 1 mg/kg to about 250 mg/kg, and may given in a single dose or in two or more divided doses. Such doses may be administered in any manner useful in directing the active compounds herein to the recipient's bloodstream, including orally, via implants, parenterally (including intravenous, intraperitoneal and subcutaneous injections), rectally, vaginally, and transdermally. For the purposes of this disclosure, transdermal administrations are understood to include all administrations across the surface of the body and the inner linings of bodily passages including epithelial and mucosal tissues. Such administrations may be carried out using the present compounds, or pharmaceutically acceptable salts thereof, in lotions, creams, foams, patches, suspensions, solutions, and suppositories (rectal and vaginal).

Oral formulations containing the active compounds of this invention may comprise any conventionally used oral forms, including tablets, capsules, buccal forms, troches, lozenges and oral liquids, suspensions or solutions. Capsules may contain mixtures of the active compound(s) with inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc. Useful tablet formulations may be made by conventional compression, wet granulation or dry granulation methods and utilize pharmaceutically acceptable diluents, binding agents, lubricants, disintegrants, suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Oral formulations herein may utilize standard delay or time release formulations to alter the absorption of the active compound(s). Suppository formulations may be made from traditional materials, including cocoa butter, with or without the addition of waxes to alter the suppository's melting point, and glycerin. Water soluble suppository bases, such as polyethylene glycols of various molecular weights, may also be used.

The sulfonylurea agents of this invention may be administered at doses and regimens known in the art, such as those listed for the relevant compounds in the Physicians' Desk Reference, 55 Edition, 2001, published by Medical Economics Company, Inc. at Montvale, N.J. For example, glimepiride, which is available in AMARYL® tablets from Aventis Pharmaceuticals, may be given at an initial daily dosage of from about 1 to about 2 mg per day in human adults. This dosage may be increased gradually up to about 8 mg per day, with a usual maintenance dose being between about 2 and 4 mg per day. Glyburide is available in DIABETA® tablets from Aventis Pharmaceuticals, and has an initial dose ranging from about 2.5 to about 5 mg per day and a usual maintenance dose of from about 1.25 to about 20 mg per day. Chlorpropamide is available from Pfizer Inc. in DIABINESE® tablets, and may have a daily dose in humans of from about 100 to about 500 mg, depending upon the individual characteristics of the recipient. Glipizide is commercially available in GLUCOTROL® tablets and GLUCOTROL XL® extended release tablets from Pfizer Inc. It can be administered at an initial daily dose of from about 2.5 to about 5 mg and increased in 2.5 to 5 mg increments to a maintenance dose of between about 15 and 40 mg per day. Tolazamide is generally administered at a daily dosage of between about 100 mg and 500 mg per day, with an average maintenance dose of between about 250 mg and 500 mg per day taken once daily or divided into multiple administrations over the course of a day. 250 mg and 500 mg tablets of tolazamide and 500 mg tablets of tolbutamide are available from Mylan Pharmaceuticals Inc., Morgantown, W.Va., U.S.A.

It is understood that the dosage, regimen and mode of administration of these compounds will vary according to the malady and the individual being treated and will be subject to the judgment of the medical practitioner involved. It is preferred that the administration of one or more of the compounds herein begin at a low dose and be increased until the desired effects are achieved. It is also preferred that the recipient also utilize art recognized lifestyle patterns for reducing the incidence of the maladies described herein. These include maintenance of an appropriate diet and exercise regimen, as recommended by a medical practitioner familiar with the physical condition of the recipient.

The following are representative compound examples useful in the methods of this invention. Their synthesis is described in published PCT Application WO 99/61435, published Dec. 2, 1999, the contents of which are incorporated herein by reference.

### EXAMPLE 1

### 2,3- Dimethyl-thiophene

### EXAMPLE 2

### 4, 5-Dimethylthiophene-2-yl—(phenyl)-methanol

### EXAMPLE 3

### 2-Benzyl-4, 5 dimethylthiophene

### EXAMPLE 4

### (2-Benzyl-4, 5-dimethyl-thiophen-3-yl)—(4-methoxy-phenyl)-methanone

### EXAMPLE 5

### 4—(2, 3-Dimethyl-naphtho[2,3-b]thiophen-4-yl-phenol

### EXAMPLE 6

### Acetic Acid 4-(2, 3-dimethyl-naphtho[2,3-b]thiophen4-yl)-phenyl ester

### EXAMPLE 7

### Acetic Acid 4-(9-bromo-2, 3-dimethyl-naphtho[2,3-b]thiophen -4-yl)-phenyl ester

### EXAMPLE 8

### 4—(9-Bromo-2, 3-dimethyl- naphtho[2,3- b]thiophen-4-yl)-phenol

### EXAMPLE 9

### 2, 6-Dibromo-4-(9-bromo-2, 3-dimethyl-naphtho[2,3-b]thiophen-4-yl)-phenol

### EXAMPLE 10

### Methanesulfonic acid 4-(2,3-dimethyl-naphtho[2,3-b]thiophen-4-yl)-phenyl ester

### EXAMPLE 11

### Methanesulfonic acid 4-(9-iodo-2,3-dimethyl-naphtho[2,3-b]thiophen-4-yl)-phenyl ester

### EXAMPLE 12

### 4-(2,3-Dimethyl-9-phenylsulfanyl-naphtho[2,3-b]thiophen-4-yl)-phenol

### EXAMPLE 13

### 2,6-Dibromo-4-(2,3-dimethyl-9-phenylsulfanyl-naphtho[2,3-b]thiophen-4-yl)-phenol

### EXAMPLE 14

### Acetic acid 4-(9-bromo-2-chloromethyl-3-methyl-naphtho[2,3-b]thiophen-4-yl)-phenyl ester

### EXAMPLE 15

### 4-(9-Bromo-3-meth yl-2-morpholin-4-yl)methyl-naphtho[2,3-b]thiophen-4-yl)-phenol

### EXAMPLE 16

### 4-(9-Bromo-2-diethylaminomethyl-3-methyl-naphtho[ 2,3-b]thiophen-4-yl)-acetate

### EXAMPLE 17

### 4-(9-Bromo-2-diethylaminomethyl-3-methyl-naphtho[2,3-b]thiophen-4-yl)-phenol

### EXAMPLE 18

### 2,6-Dibromo-4-(9-bromo-2-diethylaminomethyl-3-methyl-naphtho[2,3-b]thiophen-4-yl)-phenol

### EXAMPLE 19

### 2,6-Dibromo-4-(9-bromo-3-methyl-2-morpholin-4-ylmethyl-naphtho[2,3-b]thiophen4-yl)-phenol

### EXAMPLE 20

### 4-(9-Bromo-2, 3-dimethyl-naphtho[2,3-b]thiophen-4-yl)-2-nitro-phenol

### EXAMPLE 21

### 2-Bromo-4-(9-bromo-2, 3-dimethyl-naphtho[2,3-b]thiophen-4-yl)-6-nitro-phenol

### EXAMPLE 22

### 2-Amino-4-(9-bromo-2, 3-dimethyl-naphtho[2,3-b]thiophen-4-yl)-phenol

### EXAMPLE 23

### 2-Amino-6-bromo-4-(9-bromo-2, 3-dimethyl-naphtho[2,3-b]thiophen-4-yl)-phenol

### EXAMPLE 24

### [2-Bromo-4-(9-bromo-2,3-dimethyl-naphtho[2,3-b]thiophen-4-yl)-2-nitro-phenoxy]-acetic acid

### EXAMPLE 25

### (S)-2-Hydroxy-3-phenylpropionic acid, methyl ester

### EXAMPLE 26

### (S)-2-[4-Nitrobenzoyl]-4-phenylbutyric acid, ethyl ester

### EXAMPLE 27

### (S)-2-Hydroxy-4-phenylbutyric Acid, ethyl ester

### EXAMPLE 28

### (R)-2-[2,6-Dibromo-4-(9-bromo-2,3-dimethyl-naphtho[2,3-b]thiophen-4-yl)-phenoxy]3-phenyl-propionic acid methyl ester

### EXAMPLE 29

### (R)-2-[2,6-Dibromo-4-(9-bromo-2,3-dimethyl-naphtho[2,3-b]thiophen-4-yl)-phenoxy]3-phenyl-propionic acid

### EXAMPLE 30

### (R)-2-[2,6-Dibromo-4-(9-bromo-2,3-dimethyl-naptho[2,3-b]thien-4-yl)-phenoxy]-propanoic acid

### EXAMPLE 31

### (S)-2-[2,6-Dibromo-4-(9-bromo-2,3-dimethyl-naphtho[2,3-b]thiophen-4-yl)-phenoxy]4-phenyl-butyric acid

### EXAMPLE 32

### (R)-2-[2,6-Dibromo-4-(9-bromo-2,3-dimethyl-naphtho[2,3-b]thiophen-4-yl)-phenoxy]-4-phenyl-butyric acid

### EXAMPLE 33

### (R)-2-[2,6-dibromo-4-(2,3-dimethyl-9-phenylsulfanyl-naphtho[2,3-b]-thiophen-4-yl)-phenoxy]-3-phenyl-propionic acid

### EXAMPLE 34

### (R)-2-[2,6-Dibromo-4-(2,3-dimethyl-9-phenylsulfanyl-naphtho[2,3-b]-thiophen-4-yl)-phenoxy]-propionic acid

### EXAMPLE 35

### 2-[2,6-Dibromo-4-(9-bromo-3-methyl-2-morpholin-4-ylmethyl-naphtho[2,3-b]thiophen-4-yl)-phenoxy]-3-phenyl-propionic acid

### EXAMPLE 36

### 2-[2,6-Dibromo-4-(9-bromo-3-methyl-2-morpholin-4-ylmethyl-naphtho[2,3-b]thiophen-4-yl)-phenoxy]-propionic acid

### EXAMPLE 37

### (R)-2-[2,6-Dibromo-4-(9-bromo-2-diethylaminomethyl-3-methyl-naphtho[2,3-b]thiophen-4-yl)-phenoxy]-3-phenyl-propionic acid

### EXAMPLE 38

### [2-Bromo4-(9-bromo-2, 3-dimethyl-naphtho[2,3-b]thiophen-4-yl)-2-nitro-phenoxy]-3-phenyl-propionic acid

### EXAMPLE 39

### 2-Bromo-4-(9-bromo-2, 3-dimethyl-naphtho[2,3-b]thiophen-4-yl)-6-isopropyl-phenol

### EXAMPLE 40

### (R)-2-[2-Bromo-4-(9-bromo-2, 3-dimethyl-naphtho[2,3-b]thiophen-4-yl)-6-isopropyl-phenoxy]-3-phenyl-propionic acid

### EXAMPLE 41

### (R)-2-[4-(2, 3-dimethyl-naphtho[2,3-b]thiophen-4-yl)-2-isopropyl-phenoxy]-3-phenyl-propionic acid

### EXAMPLE 42

### (R)-2-[2-Bromo-4-(9-bromo-2, 3-dimethyl-naphtho[2,3-b]thiophen-4-yl)-6-sec-butyl-phenoxy]-3-phenyl-propionic acid

### EXAMPLE 43

### (R)-2-[2-Bromo-4-(9-bromo-2, 3-dimethyl-naphtho[2,3-b]thiophen-4-yl)-6-ethyl-phenoxy]-3-phenyl-propionic acid

### EXAMPLE 44

### (R)-2-[4-(9-Bromo-2, 3-dimethyl-naphtho[2,3-b]thiophen-4-yl)-6-isopropyl-phenoxy]-3-phenyl-propionic acid

### EXAMPLE 45

### (R)-2-[2—Cyclopentyl-4-(2,3-dimethyl-naphtho[2,3-b]thiophen-4-yl)-phenoxy]-3-phenyl-propionic acid

### EXAMPLE 46

### (R)-2-[4-(2,3-Dimethyl-naphtho[2,3-b]thiophen-4-yl)-2,6-dimethyl-phenoxy]-3-phenyl-propionic acid

### EXAMPLE 47

### (R)-2-[4-(9-Bromo-2,3-dimethyl-naphtho[2,3-b]thiophen-4-yl)-2-cyclopentyl-phenoxy]-3-phenyl-propionic acid

### EXAMPLE 48

### (R)-2-[2-Bromo-4-(9-bromo-2,3-dimethyl-naphtho[2,3-b]thiophen-4-yl)-2-cyclopentyl-phenoxy]-3-phenyl-propionic acid

### EXAMPLE 49

### (R)-2-[4-(9-Bromo-2,3-dimethyl-naphtho[2,3-b]thiophen-4-yl)-2,6-dimethyl-phenoxy]-3-phenyl-propionic acid

### EXAMPLE 50

### (R)-2-[4-(2, 3-Dimethyl-naphtho[2,3-b]thiophen-4-yl)-2, 6-diisopropyl-phenoxy]-3-phenyl-propionic acid

### EXAMPLE 51

### (R)-2-[4-(2,3-Dimethyl-naphtho[2,3-b]thiophen-4-yl)-2-fluoro-phenoxy]-3-phenyl-propionic acid

### EXAMPLE 52

### (R)-2-[4-(9-Bromo-2, 3-dimethyl-naphtho[2,3-b]thiophen4-yl)-2-fluoro-phenoxy]-3-phenyl-propionic acid

### EXAMPLE 53

### [4-(9-Bromo-2, 3-dimethyl-naphtho[2,3-b]thiophen-4-yl)-2, 6-diisopropyl-phenoxy]-acetic acid

### EXAMPLE 54

### (2R)-2-[2,6-Dibromo-4-(2,3-dimethyl-naphtho[2,3-b]furan-4-yl)-phenoxy]-3-phenyl-propionic acid

### EXAMPLE 55

### (2R)-2-[4-(9-Bromo-2,3-dimethyl-naphtho[2,3-b]thiophen-4-yl)-2,6-diisopropyl-phenoxy]-3-phenyl-propionic acid

### EXAMPLE 56

### [3-Bromo-5—(9-bromo-2, 3-dimethyl-naphtho[2,3-b]thiophen-4-yl)-2-hydroxy-phenyl]-carbamic acid tert-butyl ester

### EXAMPLE 57

### 9-Bromo-4-(3-bromo-methoxy-5-nitro-phenyl)-2, 3-dimethyl-naphtho[2,3-b]thiophene

### EXAMPLE 58

### 3-Bromo-5—(9-bromo-2,3-dimethyl-naphtho[2,3-b]thiophen-4-yl)-2-methoxy-phenylamine

### EXAMPLE 59

### [3-Bromo-5—(9-bromo-2,3-dimethyl-naphtho[2,3-b]thiophen-4-yl)-2-methoxy-phenylamino]-acetic acid methyl ester

### EXAMPLE 60

### [3-Bromo-5—(9-bromo-2,3-dimethyl-naphtho[2,3-b]thiophen-4-yl)-2-methoxy-phenylamino]-acetic acid

### EXAMPLE 61

### (R)-2-[4-(9-Bromo-2-,3-dimethyl-naphtho[2,3-b]thiophen-4-yl)-2,6-diethyl-phenoxy]-3-phenyl-propionic acid

### EXAMPLE 62

### {(2R)-2-[4-(9-Bromo-2,3-dimethyl-naphtho[2,3-b]thiophen-4-yl)-2,6-dimethyl-phenoxy]-3-phenyl-propionylamino}-acetic acid

### EXAMPLE 63

### {(2R)-2-[4-(9-Bromo-2,3-dimethyl-naphtho[2,3-b]thiophen-4-yl)-2,6-diethyl-phenoxy]-3-phenyl-propionylamino}-acetic acid

### EXAMPLE 64

### (2R)-2-[4-(9-Bromo-2,3-dimethyl-naphtho[2,3-b]thiophen-4-yl)-phenoxy]-3-phenyl-propionic acid

### EXAMPLE 65

### (2S)-2-[4(9-Bromo-2-,3-dimethyl-naphtho[2,3-b]thiophen-4-yl)-2,6-dimethyl-phenoxy]-3-phenyl-propionic acid

### EXAMPLE 66

### (2R)-2-[4(9-Bromo-2,3-dimethyl-1-oxo-1H-naphtho[2,3-b]thiophen-4-yl)-2,6-dimethyl-phenoxy]-3-phenyl-propionic acid

### EXAMPLE 67

### (R)-2-[4-(2-,3-Dimethyl-naphtho[2,3-b]thiophen-4-yl)-2,6-diethyl-phenoxy]-3-phenyl-propionic acid

### EXAMPLE 68

### {(2R)-2-[4-(2,3-Dimethyl-naphtho[2,3-b]thiophen-4-yl)-2,6-diethyl-phenoxy]-3-phenyl-propionylamino}-acetic acid

### EXAMPLE 69

### 4-(2,3-Dimethyl-naphtho[2,3-b]furan-4-yl)-2,6-diethyl-phenol

### EXAMPLE 70

### (R)-2-[4(9-Bromo-2-,3-dimethyl-naphtho[2,3-b]furan-4-yl)-2,6-diethyl phenoxy]-3-phenyl-propionic acid

### EXAMPLE 71

### (R)-2-[2—Cyclopentyl-4-(2-,3-dimethyl-naphtho[2,3-b]thiophen-4-yl)-phenoxy]-propionic acid

### EXAMPLE 72

### (R)-2-[4-(9-Bromo-2-,3-dimethyl-naphtho[2,3-b]thiophen-4-yl)-2-cyclopentyl-phenoxy]-propionic acid

### EXAMPLE 73

### 4-[4-(9-Bromo-2-,3-dimethyl-naphtho[2,3-b]thiophen-4-yl)-2-cyclopentyl-phenoxy]-butyric acid

### EXAMPLE 74

### 2-Cyclopentyl-4-(2-,3-dimethyl-naphtho[2,3-b]furan-4-yl)-phenol

### EXAMPLE 75

### Acetic acid 2-cyclopentyl-4-(2-,3-dimethyl-naphtho[2,3-b]furan-4-yl)-phenyl ester

### EXAMPLE 76

### (R)-2-[4-(2-,3-Dimethyl-naphtho[2,3-b]thiophen-4-yl)-2-ethyl-phenoxy]-3-phenyl-propionic acid

### EXAMPLE 77

### (R)-2-[4-(9-Bromo-2-,3-dimethyl-naphtho[2,3-b]thiophen-4-yl)-2-ethyl-phenoxy]-3-phenyl-propionic acid

### EXAMPLE 78

### 2-Bromo-4-(2-,3-dimethyl-naphtho[2,3-b]furan-4-yl)-6-ethyl-phenol

### EXAMPLE 79

### (R)-2-[2-Bromo-4-(2-,3-dimethyl-naphtho[2,3-b]furan-4-yl)-6-ethyl-phenoxy]-3-phenyl-propionic acid

### EXAMPLE 80

### 4-[2-Bromo-4-(2,3-dimethyl-naphtho[2,3-b]furan4-yl)-6-ethyl-phenoxy]-butyric acid

### EXAMPLE 81

### 4-[2-Bromo-4-(2,3-dimethyl-naphtho[2,3-b]furan-4-yl)-6-ethyl-phenoxy]-butyramide 0.4 hydrate

### EXAMPLE 82

### 4-(2,3-Dimethyl-naphtho[2,3-b]furan-4-yl)-2-ethyl-phenol

### EXAMPLE 83

### (R)-2-[4(9-Bromo-2-,3-dimethyl-naphtho[2,3-b]thiophen-4-yl)-2-propyl-phenoxy]-3-phenyl-propionic acid

### EXAMPLE 84

### [9-Bromo-4-(4-methoxy-3,5-dimethylphenyl)-3-methylnaphtho[2,3-b]thien-2-yl]methyl acetate

### EXAMPLE 85

### 4-(9-Bromo-2-,3-dimethyl-naphtho[2,3-b]thien-4-yl)-2-methyl-phenyl acetate

### EXAMPLE 86

### Acetic acid 4-(9-bromo-2-diethylaminomethyl-3-methyl-naphtho[2,3-b]-thiophen-4-yl)-2,6-dimethyl-phenyl ester

### EXAMPLE 87

### 2-[4-(9-Bromo-2-diethylaminomethyl-3-methyl-naphtho[2,3-b]thiophen-4-yl)-2,6-dimethyl-phenoxy]-3-phenyl-propionic acid; and

### EXAMPLE 88

### (2R)-2-[4-(9-Bromo-2-diethylaminomethyl-3-methyl-naphtho[2,3-b]thiophen-4-yl)-2,6-diisopropyl-phenoxy]-3-phenyl-propionic acid

or the pharmaceutically acceptable salt or ester forms thereof.

What is claimed:

1. A method for improving the cardiovascular risk profile in mammals experiencing or subject to Syndrome X or type II diabetes, the method comprising administering to a mammal in need thereof a pharmaceutically effective amount of a sulfonylurea agent and a pharmaceutically effective amount of a protein-tyrosine phosphatase inhibiting compound of the formula: 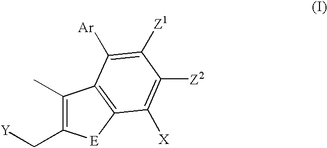
wherein
Ar is 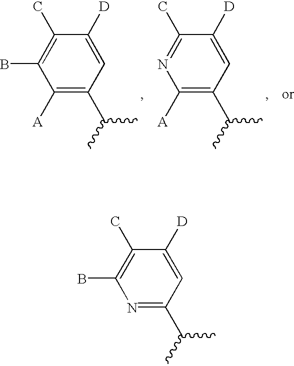
A is hydrogen, halogen, or OH; B and D are each, independently, hydrogen, halogen, CN, alkyl of 1-6 carbon atoms, aryl, aralkyl of 6-12 carbon atoms, hydroxyalkyl of 1-6 carbon atoms, hydroxyaralkyl of 6-12 carbon atoms, cycloalkyl of 3-8 carbon atoms, nitro, amino, —NR¹R^{1a}, —NR¹COR^{1a}, —NR¹CO₂R^{1a}, cycloalkylamino of 3-8 carbon atoms, morpholino, furan-2-yl, furan-3-yl, thiophen-2-yl, thiophen-3-yl, —COR^{1b} or OR;
R is hydrogen, alkyl of 1-6 carbon atoms, —COR¹, —(CH₂)ₙCO₂R¹, —CH(R^{1a}) CO₂R¹, —SO₂R¹, (CH₂)ₘCH(OH)CO₂R¹, (CH₂)ₘCOCO₂R¹, —(CH₂)ₘCH═CHCO₂R¹, —(CH₂)ₘO(CH₂)ₒCO₂R¹;
R¹ is hydrogen, alkyl of 1-6 carbon atoms, aralkyl of 6-12 carbon atoms, aryl, or CH₂CO₂R¹;
R^{1′}is hydrogen or alkyl of 1-6 carbon atoms
E is S, SO₂, SO, O, or NR^{1c};
X is hydrogen, halogen, alkyl of 1-6 carbon atoms, alkenyl of 2-7 carbon atoms, CN, aryl, aralkyl of 6-12 carbon atoms, hydroxyalkyl of 1-6 carbon atoms, hydroxyaralkyl of 6-12 carbon atoms, perfluoroalkyl of 1-6 carbon atoms, alkoxy of 1-6 carbon atoms, aryloxy; arylalkoxy, nitro, amino, NR²R^{2a}, NR²COR^{2a}, cycloalkylamino of 3-8 carbon atoms, morpholino, alkylsulfanyl of 1-6 carbon atoms, arylsulfanyl, pyridylsulfanyl, 2-N,N-dimethylaminoethylsulfanyl, —OCH₂CO₂R^{2b} or —COR^{2c};
Y is hydrogen, halogen, alkyl of 1-6 carbon atoms, aryl, aralkyl of 6-12 carbon atoms, hydroxyalkyl of 1-6 carbon atoms, hydroxyaralkyl of 6-12 carbon atoms, —OR³, SR³, NR³R^{3a}, —COR^{3b}, morpholine or piperidine;
R^{1a}, R^{1c}, R², R^{2a} R³, R^{3a} are each, independently, hydrogen, alkyl of 1-6 carbon atoms, aralkyl of 6-12 carbon atoms, or aryl;
R^{1b} is alkyl of 1-6 carbon atoms or aryl;
R^{2b} is hydrogen, alkyl of 1-6 carbon atoms;
R^{2c} and R^{3b} are each, independently, alkyl of 1-6 carbon atoms, aryl, or aralkyl of 6-12 carbon atoms;
C is hydrogen, halogen or OR⁴;
R⁴ is hydrogen, alkyl of 1-6 carbon atoms, —CH(R₅)W, —C(CH₃)₂CO₂R⁶, 5-thiazolidine-2,4-dione, —CH(R⁷)(CH₂)ₘCO₂R⁶, —COR⁶, —PO₃(R⁶)₂, —SO₂R⁶, —(CH₂)ₚCH(OH)CO₂R⁶, —(CH₂)ₚCOCO₂R⁶, —(CH₂)ₚCH═CHCO₂R⁶, or —(CH2)ₚO(CH₂)_{q}CO₂R⁶;
R⁵ is hydrogen, alkyl of 1-6 carbon atoms, aralkyl of 6-12 carbon atoms, aryl, —CH₂(1H-imidazol-4-yl), —CH₂(3-1H-indolyl), —CH₂CH₂(1,3-dioxo-1,3-dihydro-isoindol-2-yl), —CH₂CH₂(1-oxo-1,3-dihydro-isoindol-2-yl), —CH₂(3-pyridyl), —CH₂CO₂H, or —(CH₂)ₙG;
G is NR^{6a}R^{7a}, NR^{6a}COR^{7a} 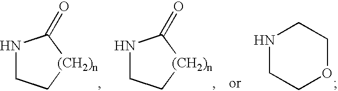
W is CO₂R⁶, CONH₂, CONHOH, CN, CONH(CH₂)₂CN, 5-tetrazole, —PO₃(R⁶)₂, —CH₂OH, —CONR^{6b}CHR^{7b}, —CH₂NR^{6b}CHR^{7b}CO₂R⁶, —CH₂OCHR^{7b}CO₂R⁶ —CH₂Br, or —CONR^{6b}CHR^{7b}CO₂R⁶;
R⁶, R^{6a}, R⁷, R^{7a} are each, independently, is hydrogen, alkyl of 1-6 carbon atoms, or aryl;
R^{6b} is hydrogen or —COR^{6c};
R^{6c} is alkyl of 1-6 carbon atoms or aryl;
R^{7b} is hydrogen, alkyl of 1-6 carbon atoms, or hydroxyalkyl of 1-6 carbon atoms;
Z¹ and Z² are each, independently, hydrogen, halogen, CN, alkyl of 1-6 carbon atoms, aryl, aralkyl of 6-12 carbon atoms, cycloalkyl of 3-8 carbon atoms, nitro, amino, —NR¹R^{1a}, —NR¹COR^{1a}, cycloalkylamino of 3-8 carbon atoms, morpholino, or OR⁸, or Z¹ and Z² may be taken together as a diene unit having the formula —CH═CR⁹—CR¹⁰═CR¹¹—;
R⁸ is hydrogen, alkyl of 1-6 carbon atoms, or aryl;
R⁹, R¹⁰, and R¹¹ are each, independently, hydrogen, alkyl of 1-6 carbon atoms, aryl, halogen, hydroxy, or alkoxy of 1-6 carbon atoms
m is 1 to 4
n is 1 or 2;
p is 1 to 4;
q is 1 to 4;
or a pharmaceutically acceptable salt thereof.

2. The method according to claim 1, wherein 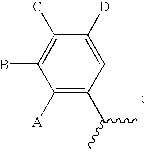
Ar is
A is hydrogen or halogen
B and D are each, independently, hydrogen, halogen, CN, alkyl of 1-6 carbon atoms, aryl, aralkyl of 6-12 carbon atoms, branched alkyl, cycloalkyl of 3-8 carbon atoms, nitro or OR;
R is hydrogen or alkyl of 1-6 carbon atoms;
E is S, or O;
X is hydrogen, halogen, alkyl of 1-6 carbon atoms, CN, perfluoroalkyl of 1-6 carbon atoms, alkoxy of 1-6 carbon atoms, aryloxy; arylalkoxy, nitro, amino, NR²R^{2a}, NR²COR^{2a}, cycloalkylamino, morpholino, alkylsulfanyl of 1-6 carbon atoms, arylsulfanyl, pyridylsulfanyl, or 2-N,N-dimethylaminoethylsulfanyl;
R¹, R^{1a}, R², R^{2a}, R³, and R^{3a} are each, independently, hydrogen, alkyl of 1-6 carbon atoms, aralkyl of 6-12 carbon atoms, or aryl;
Y is hydrogen, halogen, OR³, SR³, NR³R^{3a}, or morpholine;
C is hydrogen, halogen, or OR⁴;
R⁴ is hydrogen, alkyl of 1-6 carbon atoms, —CH(R⁵)W, —C(CH₃)₂CO₂R⁶, 5-thiazolidine-2,4-dione, —CH (R⁷)(CH₂)ₘCO₂R⁶,—COR⁶,—P₃(R⁶)₂, —SO₂R⁶, —(CH₂)ₚCH(OH)CO₂R⁶, —(CH₂)ₚCOCO₂R⁶, —(CH₂)ₚCH═CHCO₂R⁶, —(CH₂)ₚO(CH₂)_{q}CO₂R⁶;
R⁵ is hydrogen, alkyl of 1-6 carbon atoms, aralkyl of 6-12 carbon atoms, aryl, —CH₂(1H-imidazol-4-yl), —CH₂(3-1H-indolyl), —CH₂CH₂(1,3-dioxo-1,3-dihydro-isoindol-2-yl), —CH₂CH₂(1-oxo-1,3-dihydro-isoindol-2-yl), or —CH₂(3-pyridyl);
W is CO₂R⁶, —CONH₂, —CONHOH, 5-tetrazole, or —CONR^{6b}CHR^{7b}CO₂R⁶;
R⁶, R^{6a}, R^{6b}, R⁷, R⁷a , and R^{7b} are each, independently, hydrogen, alkyl of 1-6 carbon atoms, or aryl;
Z¹ and Z² are each, independently, hydrogen, halogen, CN, alkyl of 1-6 carbon atoms, aryl, aralkyl of 6-12 carbon atoms, cycloalkyl of 3-8 carbon atoms, nitro, amino, —NR¹R^{1a}, —NR¹COR^{1a}, cycloalkylamino of 3-8 carbon atoms, morpholino, or OR⁸, or Z¹ and Z² may be taken together as a diene unit having the formula —CH═CR⁹—CR¹⁰═CH—;
R⁹ and R¹⁰ are each, independently, hydrogen, or alkyl of 1-6 carbon atoms;
p is 1 to4;
q is 1 to4;
or a pharmaceutically acceptable salt thereof.

3. The method according to claim 2, wherein
A is hydrogen;
B and D are each, independently, halogen, alkyl of 1-6 carbon atoms, aryl, aralkyl of 6-12 carbon atoms, or cycloalkyl of 3-8 carbon atoms;
E is S or O;
X is hydrogen, halogen, alkyl of 1-6 carbon atoms, perfluoroalkyl of 1-6 carbon atoms, CN, alkoxy of 1-6 carbon atoms, aryloxy, arylalkoxy of 6-12 carbon atoms, arylsulfanyl;
Y is hydrogen, —NR¹R², or morpholine;
R¹ and R² are each, independently, hydrogen or alkyl of 1-6 carbon atoms, aralkyl of 6-12 carbon atoms, or aryl;
C is OR⁴;
R⁴ is hydrogen, alkyl of 1-6 carbon atoms, —CH(R⁵)W, or 5-thiazolidine-2,4-dione;
R⁵ is hydrogen, alkyl of 1-6 carbon atoms, aralkyl of 6-12 carbon atoms, aryl, —CH₂(3-1H-indolyl), —CH₂CH₂(1,3-dioxo-1,3-dihydro-isoindol-2-yl), or —CH₂CH₂(1-oxo-1,3-dihydro-isoindol-2-yl);
W is —CO₂R⁶, —CONH₂, —CONHOH, 5-tetrazole, —PO₃(R⁶)₂, or —
CONR⁶CHR⁶CO₂R⁶;
R⁶ is hydrogen or alkyl of 1-6 carbon atoms;
Z¹ and Z² are taken together as a diene unit having the formula —CH═CH—H═CH—;
or a pharmaceutically acceptable salt thereof.

4. A method according to claim 1 wherein the protein-tyrosine phosphatase inhibiting compound is selected from the group of
(R)-2-[2,6-dibromo-4-(9-bromo-2,3-dimethyl-naphtho[2,3-b]thiophen-4-yl)-phenoxy]-3-phenyl-propionic acid;
(R)-2-[2-bromo-4-(9-bromo-2, 3-dimethyl-naphtho[2,3-b]thiophen-4-yl)-6-ethyl-phenoxy]-3-phenyl-propionic acid;
(R)-2-[4-(9-bromo-2, 3-dimethyl-naphtho[2,3-b]thiophen-4-yl)-2, 6-dimethyl-phenoxy]-3-phenyl-propionic acid;
(R)-2-[4-(9-bromo-2, 3-dimethyl-naphtho[2,3-b]thiophen-4-yl)-2-fluoro-phenoxy]-3-phenyl-propionic acid; or
[4-(9-bromo-2, 3-dimethyl-naphtho[2,3-b]thiophen-4-yl)-2, 6-diisopropyl-phenoxy]-acetic acid; or a pharmaceutically acceptable salt or ester form thereof.

5. A method according to claim 1 wherein the protein-tyrosine phosphatase inhibiting compound is selected from the group of:
(R)-2-[2-bromo-4-(9-bromo-2, 3-dimethyl-naphtho[2,3-b]thiophen-4-yl)-6-sec-butyl phenoxy]-3-phenyl-propionic acid;
(R)-2-[2-bromo-4-(9-bromo-2, 3-dimethyl-naphtho[2,3-b]thiophen-4-yl)-6-isopropyl-phenoxy]-3-phenyl-propionic acid;
(R)-2-[2-bromo-4-(9-bromo-2, 3-dimethyl-naphtho[2,3-b]thiophen-4-yl)-2-cyclopentyl-phenoxy]-3-phenyl-propionic acid;
(R)-2-[4-(9-bromo-2, 3-dimethyl-naphtho[2,3-b]thiophen-4-yl)-6-isopropyl-phenoxy]-3-phenyl-propionic acid; or
(R)-2-[4-(9-bromo-2, 3-dimethyl-naphtho[2,3-b]thiophen-4-yl)-2-cyclopentyl-phenoxy]-3-phenyl-propionic acid; or a pharmaceutically acceptable salt or ester form thereof.

6. A method according to claim 1 wherein the protein-tyrosine phosphatase inhibiting compound is selected from the group of:
(R)-2-[2,6-dibromo-4-(2,3-dimethyl-9-phenylsulfanyl-naphtho[2,3-b]thiophen-4-yl)-phenoxy]-3-phenyl-propionic acid;
(R)-2-[2,6-dibromo-4-(9-bromo-2,3-dimethyl-naphtho[2,3-b]thiophen-4-yl)-phenoxy]-4-phenyl-butyric acid;
(S)-2-[2,6-dibromo-4-(9-bromo-2,3-dimethyl-naphtho[2,3-b]thiophen-4-yl)-phenoxy]-4-phenyl-butyric acid;
2-[2,6-dibromo-4-(9-bromo-3-methyl-2-morpholin-4-ylmethyl-naphtho[2,3-b]thiophen-4-yl)-phenoxy]-3-phenyl-propionic acid; or
(R)-2-[2,6-dibromo-4-(2,3-dimethyl-9-phenylsulfanyl-naphtho[2,3-b]thiophen-4-yl)-phenoxy]-propionic acid; or a pharmaceutically acceptable salt or ester form thereof.

7. A method according to claim 1 wherein the protein-tyrosine phosphatase inhibiting compound is selected from the group of:
[2-bromo-4-(9-bromo-2, 3-dimethyl-naphtho[2,3-b]thiophen-4-yl)-2-nitro-phenoxy]-3-phenyl-propionic acid;
2, 6-dibromo-4-(9-bromo-2, 3-dimethyl-naphtho[2,3-b]thiophen-4-yl)-phenol;
2-bromo-4-(9-bromo-2, 3-dimethyl-naphtho[2,3-b]thiophen-4-yl)-6-nitro-phenol;
(R)-2-[2,6-dibromo-4-(9-bromo-2-diethylaminomethyl-3-methyl-naphtho[2,3-b]thiophen-4-yl)-phenoxy]-3-phenyl-propionic acid;
(R)-2-[2, 6-dibromo-4-(2, 3-dimethyl-naphtho[2,3-b]furan-4-yl)-phenoxy]-3-phenyl-propionic acid; or a pharmaceutically acceptable salt or ester form thereof.

8. A method according to claim 1 wherein the protein-tyrosine phosphatase inhibiting compound is selected from the group of:
(2R)-2-[4-9-bromo-2,3-dimethyl-naphtho[2,3-b]thiophen-4-yl)-2,6-diisopropyl-phenoxy]-3-phenyl-propionic acid;
(R)-2-[4-(9-bromo-2-,3-dimethyl-naphtho[2,3-b]thiophen-4-yl)-2,6-diethyl-phenoxy]-3-phenyl-propionic acid;
{(2R)-2-[4-(9-bromo-2,3-dimethyl-naphtho[2,3-b]thiophen-4-yl)-2,6-dimethyl-phenoxy]-3-phenyl-propionylamino}-acetic acid;
{(2R)-2-[4-(9-bromo-2,3-dimethyl-naphtho[2,3-b]thiophen-4-yl)-2,6-diethyl-phenoxy]-3-phenyl-propionylamino}-acetic acid;
(2R)-2-[4-(9-Bromo-2,3-dimethyl-naphtho[2,3-b]thiophen-4-yl)-phenoxy]-3-phenyl-propionic acid; or a pharmaceutically acceptable salt or ester form thereof.

9. A method of claim 1 wherein the protein-tyrosine phosphatase inhibiting compound is selected from the group of:
(2S)-2-[4-(9-Bromo-2-,3-dimethyl-naphtho[2,3-b]thiophen-4-yl)-2,6-dimethyl-phenoxy]-3-phenyl-propionic acid;
{(2R)-2-[4-(2,3-Dimethyl-naphtho[2,3-b]thiophen-4-yl)-2,6-diethyl-phenoxy]-3-phenyl-propionylamino}-acetic acid;
(R)-2-[4-(9-Bromo-2-,3-dimethyl-naphtho[2,3-b]furan-4-yl)-2,6-diethyl-phenoxy]-3-phenyl-propionic acid;
(R)-2-[2—Cyclopentyl-4-(2-,3-dimethyl-naphtho[2,3-b]thiophen-4-yl)-phenoxy]-propionic acid;
(R)-2-[4-(9-Bromo-2-,3-dimethyl-naphtho[2,3-b]thiophen-4-yl)-2-cyclopentyl-phenoxy]-propionic acid; or a pharmaceutically acceptable salt or ester form thereof.

10. A method of claim 1 wherein the protein-tyrosine phosphatase inhibiting compound is selected from the group of:
(R)-2-[4-(2-,3-Dimethyl-naphtho[2,3-b]thiophen-4-yl)-2-ethyl-phenoxy]-3-phenyl-propionic acid;
2-Bromo-4-(2-,3-dimethyl-naphtho[2,3-b]furan-4-yl)-6-ethyl-phenol;
(R)-2-[2-Bromo-4-(2-,3-dimethyl-naphtho[2,3-b]furan-4-yl)-6-ethyl-phenoxy]-3-phenyl-propionic acid;
(R)-2-[4-(9-Bromo-2-,3-dimethyl-naphtho[2,3-b]thiophen-4-yl)-2-propyl-phenoxy]-3-phenyl-propionic acid;
(2R)-2-[4-(9-Bromo-2-diethylaminomethyl-3-methyl-naphtho[2,3-b]thiophen-4-yl)-2,6-diisopropyl-phenoxy]-3-phenyl-propionic acid; or a pharmaceutically acceptable salt or ester form thereof.

11. A method of claim 1 wherein the sulfonylurea agent is selected from the group of glipizide, glyburide, chlorpropamide, tolbutamide, tolazamide, or glimepriride, or a pharmaceutically acceptable salt form thereof.

12. A method of claim 1 comprising lowering a blood lipoprotein level in a mammal experiencing or subject to Syndrome X or type II diabetes, the method comprising administering to a mammal in need thereof a pharmaceutically effective amount of a protein-tyrosine phosphatase inhibiting compound and a pharmaceutically effective amount of a sulfonylurea agent.

13. A method of claim 12 wherein the blood lipoprotein is low density lipoprotein.

14. A method of claim 1 comprising lowering a blood triglyceride level in a mammal experiencing or subject to Syndrome X or type II diabetes, the method comprising administering to a mammal in need thereof a pharmaceutically effective amount of a a protein-tyrosine phosphatase inhibiting compound and a pharmaceutically effective amount of a sulfonylurea agent.

15. A method of claim 1 comprising lowering a free fatty acid level in a mammal experiencing or subject to Syndrome X or type II diabetes, the method comprising administering to a mammal in need thereof a pharmaceutically effective amount of a protein-tyrosine phosphatase inhibiting compound and a pharmaceutically effective amount of a sulfonylurea agent.

16. A method of claim 1 comprising inhibiting atherosclerosis in a mammal experiencing or subject to Syndrome X or type II diabetes.

17. A method for lowering the cardiovascular risk profile of mammal experiencing or subject to Syndrome X or type II diabetes, the method comprising administering to a mammal in need thereof a pharmaceutically effective amount of (2R)-2-[4-(9-Bromo-2,3-dimethyl-naptho[2,3-b]thiophen-4-yl)-2,6-dimethyl-phenoxy]-3-phenyl-propionic acid, or (R)-2-[2,6-Dibromo-4-(9-bromo-2,3-dimethyl-naphtho[2,3-b]thiophen-4-yl)-phenoxy]3-phenyl-propionic acid, or (R)-2-[4-(9-Bromo-2,3-dimethyl-naphtho[2,3-b]thiophen-4-yl)-2,6-diethyl-phenoxy]-3-phenyl-propionic acid, or a pharmaceutically acceptable salt or ester form thereof, and a pharmaceutically effective amount of a sulfonylurea agent.

18. A method of claim 17 wherein the lowering of the cardiovascular risk profile of a mammal experiencing or subject to Syndrome X or type II diabetes comprises lowering a blood lipoprotein level in the mammal.

19. A method of claim 18 wherein the blood lipoprotein is low density lipoprotein.

20. A method of claim 19 wherein the lowering of the cardiovascular risk profile of a mammal experiencing or subject to Syndrome X or type II diabetes comprises lowering a blood triglyceride level in the mammal.

21. A method of claim 17 wherein the lowering of the cardiovascular risk profile of a mammal experiencing or subject to Syndrome X or type II diabetes comprises lowering a free fatty acid level in the mammal.

22. A method of claim 17 wherein the lowering of the cardiovascular risk profile of a mammal experiencing or subject to Syndrome X or type II diabetes comprises inhibiting atherosclerosis in a mammal experiencing or subject to type II diabetes.

23. A method of claim 17 wherein the sulfonylurea agent is selected from the group of glipizide, glyburide, chlorpropamide, tolbutamide, tolazamide, or glimepriride, or a pharmaceutically acceptable salt form thereof.
